(19) United States
(12) Patent Application Publication
(10) Pub. No.: US 2024/0398496 A1
(43) Pub. Date: Dec. 5, 2024

(54) SENSOR FOR AN ENDOVASCULAR ROBOTIC SYSTEM

(71) Applicant: UAB Inovatyvi Medicina, Kaunas (LT)

(72) Inventors: Vilius DAMBRAUSKAS, Kaunas (LT); Vaidas LABUNSKAS, Kaunas (LT); Giedrius KEREŽIS, Kaunas (LT); Evaldas KALVAITIS, Kaunas (LT); Edvardas SATKAUSKAS, Kaunas (LT)

(73) Assignee: UAB Inovatyvi Medicina, Kaunas (LT)

(21) Appl. No.: 18/696,955

(22) PCT Filed: Oct. 14, 2022
(86) PCT No.: PCT/EP2022/078629

§ 371 Date: Mar. 28, 2024

(30) Foreign Application Priority Data
Oct. 19, 2021 (DE) 10 2021 127 071.5

(51) Int. Cl. A61B 34/00; A61B 90/00

(52) U.S. Cl. CPC A61B 34/76; A61B 2090/064

(57) ABSTRACT

A sensor (100) for an endovascular robotic system, wherein the sensor comprises: a moveable member (6) moveable between a first position and a second position, a resilient force member (24, 24′) coupled to or integral to the moveable member (6), wherein the resilient force member (24, 24′) is configured to provide a resilient force, when the moveable member (6) is in the second position, to bias the moveable member (6) towards the first position, and a detection unit (104, 193) configured to detect a change in position of the moveable member (6) from the first position to the second position and/or the second position to the first position.

### FIELD OF THE INVENTION

The present invention generally relates to a sensor for an endovascular robotic system comprising a moveable member, a resilient force member and detection unit. The present invention further generally relates to an endovascular robotic system.

### BACKGROUND TO THE INVENTION

Endovascular specialists (for example (endo-) vascular surgeons, (interventional) cardiologists, (interventional) radiologists etc.) train, practice and develop intuitive skills to handle surgical tools. The mental imagery of skills of physicians also evolves by correlating their actions and responses of surgical tools within the human anatomy. An endovascular surgeon is generally guided by two senses: visual feedback from the imaging devices and reaction force feedback via the tool. Perception-action-visualization abilities of surgeons are fine-tuned to a level where their surgical decisions are made even without observing their hand gestures.

Currently, existing robotic systems are focused exclusively on imaging feedback, but have ignored the other source of information: tactile feedback from surgical tools. Instrument controls using a joystick and a PC interface are closer to videogame controllers than control of surgical instruments and leave vascular surgeons with less feedback information which is available performing the procedure manually.

The inventors have realized that existing vascular robotic systems are controlled via a computer interface, in contrast to what vascular surgeons are trained to do with, for example, guide wires and catheters.

There is therefore a need for improvements of (endo-) vascular robotic systems.

### SUMMARY OF THE INVENTION

The invention is set out in the independent claims. Preferred embodiments of the invention are set out in the dependent claims.

According to a first aspect, we describe a sensor for an endovascular robotic system. The sensor comprises a moveable member moveable between a first position and a second position. The sensor further comprises a resilient force member coupled to or integral to the moveable member, wherein the resilient force member is configured to provide a resilient force, when the moveable member is in the second position, to bias the moveable member towards the first position. The sensor further comprises a detection unit configured to detect a change in position of the moveable member from the first position to the second position and/or the second position to the first position.

The moveable member may allow for a user of the sensor to move the member. The moveable member may be of any suitable design. The first position and the second position may be different positions. In some examples, the moveable member is coupled to an endovascular medical instrument such as a guide wire and/or a catheter, or any other suitable instrument. In some examples, the moveable member is coupled to any suitable medical instrument. The coupling may allow the user to receive real time haptic feedback from the resilient force member.

The resilient force member may be an integral part of the moveable member and/or couplable to the moveable member. The resilient force member may provide haptic feedback to a user of the sensor. The resilient force member may be of any suitable design. In some examples, the resilient force member is configured to provide a resilient force, when the moveable member is in the second position, to bias the moveable member towards the first position. In some examples, the resilient force member is configured to provide a resilient force, when the moveable member is in the first position, to bias the moveable member towards the second position. The biasing position may be any suitable position.

The detection unit may allow for a movement of the moveable member to be detected. The detection unit may transmit the detected movements to an external source, such as, for example, a controller and/or computer and/or a server and/or a second endovascular robotic system. The detection unit may use optical means and/or magnetic field means and/or any other suitable means to detect the movement of the moveable member from the first position to the second position and/or the second position to the first position.

In some examples, the detection unit comprises an optical unit comprising a light source for emitting light and a light sensor for detecting the light emitted by the light source, wherein a first portion of the moveable member is arranged, in the first and/or second position of the moveable member, in an optical path of the emitted light between the light source and the light sensor for at least partially blocking, by the first portion of the moveable member, the emitted light travelling on the optical path between the light source and the light sensor, and wherein a first amount of the emitted light which is blockable by the first portion of the moveable member in the optical path between the light source and the light sensor is different between the moveable member being in the first position and the moveable member being in the second position, respectively. The optical unit may comprise any suitable light source and any suitable light sensor. The light sensor may be able to sense the amount of light blocked by the moveable member and/or the area in which the moveable member blocks the light—the optical unit may be configured to determine the amount of light which is blocked based on the total amount of light emitted by the light source and the amount of light which is detected by the light sensor, as will be outlined further below. The amount of light blocked by the moveable member may be in some examples different between the first and second positions and in some examples, not different. The optical unit may be able to determine the position and/or orientation of the moveable member in the optical path. In some examples, the light sensor is a linear camera with a 1500×1 pixel array. The pixel array may sense the position and/or amount of the blocked light.

In some examples, the sensor is configured to determine that the moveable member is in the first position and/or the second position based on a second amount of the emitted light, sensed by the light sensor, not blockable by the first portion of the moveable member in the optical path between the light source and the light sensor when the moveable member is in the first position and/or second position. This may allow for the sensor to determine the positioning and/or orientation of the first portion of the moveable member. This is turn may allow for the sensor to determine the inputs to the sensor made by the user.

In some examples, the sensor is configured to determine a magnitude of the resilient force based on the second amount of the emitted light sensed by the light sensor. The resilient force measured by the sensor may be the resilient force exerted by the resilient force member and/or the resilient force exerted by the user. The sensor may further comprise a processor configured to calculate the resilient force on the basis of the second amount of sensed light.

In some examples, the sensor is configured to determine that the moveable member is in a first transition between the first position and the second position and/or a second transition between the second position and the first position based on a change of a said amount of the emitted light not blockable by the first portion of the moveable member in the optical path between the light source and the light sensor. This may allow for the sensor to determine that the moveable member has been moved by the user. This may allow for the sensor to indicate any movement to the user in the case that the movement is accidental. The sensor may be able to determine the degree of transition due to the differences between the first and second amounts of sensed light.

In some examples, the resilient force is a nonlinear resilient force, and wherein the nonlinear resilient force is configured to change nonlinearly as the moveable member moves between the first position and the second position and/or between the second position and the first position. This may give the user a more realistic haptic feedback. It may also protect the sensor from damage as the moveable member is less likely to impact other parts of the sensor. This may allow for the user to have fine motor control over the moveable member.

In some examples, the resilient force is a continuous resilient force, and wherein the continuous resilient force is configured to change continuously as the moveable member moves between the first position and the second position and/or between the second position and the first position. This may give the user a more realistic haptic feedback. It may also protect the sensor from damage as the moveable member is less likely to impact other parts of the sensor. This may allow for the user to have fine motor control over the moveable member.

In some examples, the optical unit further comprises a lens arranged in the optical path between the light source and the light sensor, and wherein the lens is configured to disseminate the light emitted by the light source. This may allow for a light beam to be larger in diameter. This in turn my allow for a greater movement of the moveable member in the optical path so that the range of what can be detected is enhanced. It may also allow for the amount of blocked light to be sensed more accurately by the light sensor as the proportion of light in the optical path blocked by the moveable member is smaller. This in turn may lead to a more precise determination of the position and/or orientation of the moveable member.

In some examples, the moveable member is moveable in two degrees of freedom. This may allow for the user to have finer control of the moveable member.

In some examples, the first degree of freedom is along an axial direction of the moveable member, and wherein the second degree of freedom relates to a rotational axis about the axial direction. This may allow for the user to have finer motor control of the member and may allow for a reduction in unwanted movements of the moveable member.

In some examples, the resilient force member comprises a plurality of magnets, wherein a first magnet of the plurality of magnets is coupled to the moveable member, and wherein a second magnet of the plurality of magnets is coupled to a part of the sensor different from the moveable member, and wherein the resilient force comprises a magnetic force between the first magnet and the second magnet. This may allow for a resilient force member which provides particularly realistic haptic feedback. It may also reduce the likelihood of damage to the sensor due to overrotation and/or overmovement of the moveable member. Additionally or alternatively, springs may be used and/or any other suitable component which allows for a resilient force.

In some examples, the plurality of magnets are arranged such that, in a pair of magnets, the magnets repel or attract each other, and wherein the pair of magnets biases the moveable member towards the first position. This may allow for a resilient force member which provides particularly realistic haptic feedback. It may also reduce the likelihood of damage to the sensor due to overrotation and/or overmovement of the moveable member. The magnets may be of any suitable type and any suitable orientation.

In some examples, a distance between at least two of the plurality of magnets is configured to change based on a movement of the moveable member, and wherein the change in distance between the at least two of the plurality of magnets is configured to change the resilient force. This may allow for a resilient force member which provides particularly realistic haptic feedback.

In some examples, a first plurality of the plurality of magnets is configured to provide a change in a first directional resilient force in relation to a first degree of freedom as the moveable member is moved between the first position and the second position and/or between the second position and the first position, and wherein a second plurality of the plurality of magnets is configured to provide a change in a second directional resilient force in relation to a second degree of freedom as the moveable member is moved between the first position and the second position and/or between the second position and the first position. This may allow for a resilient force member which provides particularly realistic haptic feedback.

Additionally or alternatively, a first portion of the resilient force member is coupled to the moveable member, wherein the first portion of the resilient force member is configured to contact a first elastic component coupled to a part of the sensor different from the moveable member, and wherein the resilient force comprises a mechanical resistance force between the first portion of the resilient force member and the first elastic component. This may allow for a resilient force member which provides particularly realistic haptic feedback to a user. It may additionally or alternatively allow for a particularly cost effective method of providing haptic feedback as the first portion of the resilient force member and/or the first elastic component may comprise plastic and in particular, PETG and/or rubber. In some examples, the mechanical resistance force comprises an elastic component configured to bias the moveable member towards the first position.

In some examples, the first portion of the resilient force member and the first elastic component are arranged such that the first portion of the resilient force member and the first elastic component bias the moveable member towards the first position. This may allow for a resilient force member which provides particularly realistic haptic feedback. It may also reduce the likelihood of damage to the sensor due to overrotation and/or overmovement of the moveable member. The first portion of the resilient force member and/or the first elastic component may be of any suitable design and/or any suitable orientation.

In some examples, the first elastic member is configured to deform based on a movement of the moveable member, and wherein the deformity of the first elastic member is configured to change the resilient force. This may allow for a resilient force member which provides particularly realistic haptic feedback.

In some examples, the first portion of the resilient force member and the first elastic component are configured to provide a change in a first directional resilient force in relation to a first degree of freedom as the moveable member is moved between the first position and the second position and/or between the second position and the first position, and wherein a second portion of the resilient force member and a second elastic component are configured to provide a change in a second directional resilient force in relation to a second degree of freedom as the moveable member is moved between the first position and the second position and/or between the second position and the first position. This may allow for a resilient force member which provides particularly realistic haptic feedback.

In some examples, both the plurality of magnets and the first and/or second elastic components are used in order to provide haptic feedback.

In some examples, the light source comprises a laser diode. This may allow for a particularly effective optical unit as laser diodes provide a constant light intensity at a constant wavelength, thereby leading to a more accurate light sensor. Additionally or alternatively, any other suitable light source may be used.

In some examples, a second portion of the moveable member is arranged within an oscillation dampening pool, wherein the oscillation dampening pool is configured to provide oscillation dampening to the moveable member. This may allow for a reduction in unwanted and/or accidental movements of the moveable member. The oscillation dampening pool may be of any suitable design and may comprise any suitable method of oscillation dampening.

In some examples, the sensor further comprises an air bearing, wherein a third portion of the moveable member is arranged at least partially in the air bearing, and wherein the air bearing is configured to allow the moveable member to move in a frictionless or substantially frictionless manner. As friction is reduced, this may allow for the user to have finer control over the moveable member. The bearing may be any suitable type of bearing.

In some examples, the sensor further comprises a zero positioning unit, wherein the zero positioning unit comprises a positioning sensor and an indicator configured to indicate that the sensor is in a zero position, wherein the moveable member does not encounter a net force in the zero position. The zero position may be any suitable position. In some examples, the zero positioning unit comprises a photodiode and a light emitting diode which is covered when a zero position flag travels between them. This may then result in the light emitting diode being turned on or off, thereby indicating the moveable member is in the zero position. The light emitting diode may be turned on or off via any suitable method. In some examples, this may be used to determine the zero rotational position of a housing which surrounds the sensor. Any suitable method of indicating the zero position may be used.

In some examples, the sensor further comprises a sensor housing and a sensor bearing coupled to the sensor housing, and wherein the sensor bearing is configured to allow the sensor housing to rotate about a longitudinal axis of the sensor housing. The sensor housing may protect the sensor from external factors. The sensor bearing may allow for the sensor housing to rotate and/or measure the torque exerted by the sensor housing dynamically during continuous rotation of said housing. In some examples, only the sensor housing is rotatable while the sensor itself is stationary. In some examples, the sensor is rotatable and the sensor housing is stationary. In some examples, both the sensor and the sensor housing are rotatable.

In some examples, the moveable member is coupled to or comprises a first endovascular robotic instrument. This instrument may be any suitable endovascular instrument. This may allow for the user to receive more realistic haptic feedback and/or provide a more realistic scenario for the user. This may allow for various instruments to be swapped in and out depending on the purpose of the sensor and/or the instrument.

In some examples, the sensor is configured to transmit data relating to sensed light stemming from the light source to an external receiver. The external receiver may be any suitable receiver. The external receiver may receive the data via wired and/or wireless means. In some examples, the data is transmitted via a RS232/RS485 physical connection with a proprietary protocol.

In some examples, the external receiver is comprised in a second endovascular robotic instrument controllable based on the received data. This may allow for the movement of the moveable member to be translated to a second moveable member within the second endovascular instrument. This may allow for the user to control a similar endovascular robotic instrument as the first endovascular robotic instrument. In some examples, the first and second endovascular instruments are located at different locations.

In some examples, the sensor further comprises a slip ring, wherein the slip ring is configured to allow for substantially continuous transmission of data corresponding to the sensed light to the external receiver. This may allow for data to be transmitted constantly between the rotatable sensor housing and the sensor and/or any other external component.

In some examples, the second endovascular robotic instrument is identical or substantially identical to the first endovascular robotic instrument, and/or wherein a function of the second endovascular robotic instrument is identical to a function of the first endovascular robotic instrument (such functioning being, for example, the functioning of a catheter and/or of a guide wire). This may allow for the user to control operations at an external location and allow for operations to be translated to the second endovascular robotic instrument.

In some examples, the detection unit comprises magnetic field measurement unit configured to detect a change in a magnetic field, wherein in the first and/or second position of the moveable member, a characteristic of the magnetic field is detectable by the magnetic field measurement unit, and wherein the characteristic of the magnetic field detectable by the magnetic field measurement unit is a first value when the moveable member is in the first position and a second value when the moveable member is in the second position, wherein the first value is different from the second value.

In some examples, the magnetic field measurement unit comprises a first magnet.

The magnetic field measurement unit may comprise any suitable magnet type. The magnetic field measurement unit may be able to sense a change in a characteristic of a magnetic field in an area immediately surrounding the magnetic field measurement unit or at the location of the magnetic field measurement unit. The magnetic field measurement unit may be configured to determine the change in the characteristic of the magnetic field, as will be outlined further below. The characteristic of the magnetic field may be in some examples different between the first and second positions and in some examples, not different. The magnetic field measurement unit may be able to determine the position and/or orientation of the moveable member due to the detected characteristic. In some examples, only one characteristic of the magnetic field is detected. In some examples, a plurality of characteristics of the magnetic field are detected.

Throughout the present disclosure, the one or more characteristics of the magnetic field detectable by the magnetic field measurement unit may comprise a magnitude and/or a direction of the magnetic field.

In some examples, the sensor is configured to determine that the moveable member is in the first position and/or the second position based on the characteristic detectable by the magnetic field measurement unit when the moveable member is in the first position and/or second position. This may allow for the sensor to determine the positioning and/or orientation of the first portion of the moveable member. This is turn may allow for the sensor to determine the inputs to the sensor made by the user.

In some examples, the sensor is configured to determine a magnitude of the resilient force based on the characteristic detectable by the magnetic field measurement unit when the moveable member is in the second position. The resilient force measured by the sensor may be the resilient force exerted by the resilient force member and/or the resilient force exerted by the user. The sensor may further comprise a processor configured to calculate the resilient force on the basis of the detected characteristic of the magnetic field.

In some examples, the sensor is configured to determine that the moveable member is in a first transition between the first position and the second position and/or a second transition between the second position and the first position based on a change of the characteristic detectable by the magnetic field measurement unit. This may allow for the sensor to determine that the moveable member has been moved by the user. This may, in some examples, allow for the sensor to indicate any movement to the user in the case that the movement is accidental. The sensor may be able to determine the degree of transition due to the differences between the first and second detected characteristics of the magnetic field.

In some examples, the magnetic field measurement unit is configured to transmit data about the detected characteristic to a microcontroller coupled to the magnetic field measurement unit. This may allow for the changes in the detected characteristic of the magnetic field to be calculated. This may lead to the position and/or orientation of the moveable member to be accurately calculated. The microcontroller may comprise a transmitter which is configured to transmit the data to an external device such as, for example, a second controller, a computer or a server.

In some examples, the magnetic field measurement unit comprises a Hall effect sensor. The Hall effect sensor may be able to detect a change in a characteristic of the magnetic field if another magnet moves in relation to the Hall effect sensor (or in relation to the first magnet). In some examples, the Hall effect sensor is stationary and the other magnet is moveable, or vice versa. In some examples, both the Hall effect sensor and the other magnet are moveable.

In some examples, a second magnet is coupled to the moveable member, and wherein the characteristic detectable by the magnetic field measurement unit is based on a magnetic field interaction between the first magnet and the second magnet. This may allow for an accurate determination of the position and/or orientation of the moveable member. Changes in magnetic fields may then be detected by one or more Hall effect sensors.

In some examples, the sensor may comprise an optical unit and a magnetic field measurement unit in order to sense the position and/or the orientation of the moveable member.

According to a related aspect, we describe a sensor for an endovascular robotic system. The sensor comprises a moveable member moveable between a first position and a second position. The sensor further comprises an optical unit comprising a light source for emitting light and a light sensor for detecting the light emitted by the light source, wherein a portion of the moveable member is arranged, in the first and/or second position of the moveable member, in an optical path of the emitted light between the light source and the light sensor for at least partially blocking, by the portion of the moveable member, the emitted light travelling on the optical path between the light source and the light sensor, and wherein a first amount of the emitted light which is blockable by the portion of the moveable member in the optical path between the light source and the light sensor is different between the moveable member being in the first position and the moveable member being in the second position, respectively, and wherein the light sensor is configured to determine that the moveable member is in the first position and/or the second position based on a second amount of the emitted light, sensed by the light sensor, not blockable by the portion of the moveable member in the optical path when the moveable member is in the first position and/or second position.

The moveable member may allow for a user of the sensor to move the member. The moveable member may be of any suitable design. The first position and the second position may be different positions. In some examples, the moveable member is coupled to an endovascular medical instrument such as a guide wire, a catheter, or any other suitable instrument. In some examples, the moveable member is coupled to any suitable medical instrument. The coupling may allow for the user to receive real time haptic feedback from the resilient force member.

The optical unit may comprise any suitable light source and any suitable light sensor. The light sensor may be able to sense the amount of light blocked by the moveable member and/or the area in which the moveable member blocks the light (the optical unit may be configured to determine the amount of light which is blocked based on the total amount of light emitted by the light source and the amount of light which is detected by the light sensor). The amount of light blocked by the moveable member may be in some examples different between the first and second positions, and in some examples not different. The optical unit may be able to determine the position and/or orientation of the moveable member in the optical path. In some examples, the light sensor is a linear camera with a 1500×1 pixel array. The pixel array may sense the position and/or amount of the blocked light.

According to a further aspect, we describe a sensor for an endovascular robotic system, wherein the sensor comprises a moveable member moveable between a first position and a second position, and a magnetic field measurement unit comprising a first magnet for detecting changes in a magnetic field, wherein in the first and/or second position of the moveable member, a characteristic of the magnetic field is detectable by the magnetic field measurement unit, wherein the characteristic of the magnetic field detectable by the magnetic field measurement unit is a first value when the moveable member is in the first position and a second value when the moveable member is in the second position, wherein the first value is different from the second value, and wherein the sensor is configured to determine that the moveable member is in the first position and/or the second position based on the characteristic detectable by the magnetic field measurement unit when the moveable member is in the first position and/or second position.

The moveable member may allow for a user of the sensor to move the member. The moveable member may be of any suitable design. The first position and the second position may be different positions. In some examples, the moveable member is coupled to an endovascular medical instrument such as a guide wire and/or a catheter, or any other suitable instrument. In some examples, the moveable member is coupled to any suitable medical instrument. The coupling may allow the user to receive real time haptic feedback from the resilient force member.

The resilient force member may be an integral part of the moveable member and/or couplable to the moveable member. The resilient force member may provide haptic feedback to a user of the sensor. The resilient force member may be of any suitable design. In some examples, the resilient force member is configured to provide a resilient force, when the moveable member is in the second position, to bias the moveable member towards the first position. In some examples, the resilient force member is configured to provide a resilient force, when the moveable member is in the first position, to bias the moveable member towards the second position. The biasing position may be any suitable position.

The magnetic field measurement unit may comprise any suitable magnet type. The magnetic field measurement unit may be able to sense a change in a characteristic of a magnetic field in an area immediately surrounding the magnetic field measurement unit or at the location of the magnetic field measurement unit. The magnetic field measurement unit may be configured to determine the change in the characteristic of the magnetic field, as will be outlined further below. The characteristic of the magnetic field may be in some examples different between the first and second positions and in some examples, not different. The magnetic field measurement unit may be able to determine the position and/or orientation of the moveable member due to the detected characteristic. In some examples, only one characteristic of the magnetic field is detected. In some examples, a plurality of characteristics of the magnetic field are detected.

The sensor is configured to determine that the moveable member is in the first position and/or the second position based on the characteristic detected by the magnetic field measurement unit when the moveable member is in the first position and/or second position. This may allow for the sensor to determine the positioning and/or orientation of the first portion of the moveable member. This is turn may allow for the sensor to determine the inputs to the sensor made by the user. The characteristic may be, for example, the presence and/or the magnitude and/or the direction of the magnetic field surrounding the magnetic field measurement unit or at the location of the magnetic field measurement unit.

According to a further aspect, we describe an endovascular robotic system. The endovascular robotic system comprises a first endovascular robotic instrument located at a first location, and a second endovascular robotic instrument located at a second location different from the first location, wherein the first endovascular robotic instrument is communicatively coupled with the second endovascular robotic instrument, and wherein the first endovascular robotic instrument and/or the second endovascular robotic instrument comprises the sensor as described in one or more of the above aspects and examples of the present specification.

In some examples, a first functioning of the first endovascular robotic instrument is identical to a second functioning of the second endovascular robotic instrument, wherein the first endovascular robotic instrument comprises a first haptic feedback unit configured to generate first haptic feedback data dependent on a first movement, for implementing the first functioning, of the first endovascular robotic instrument, wherein the first endovascular robotic instrument is configured to send the first haptic feedback data to the second endovascular robotic instrument, and wherein the second endovascular robotic instrument is configured to mimic, for implementing the second functioning, the first movement of the first endovascular robotic instrument based on the first haptic feedback data received from the first endovascular robotic instrument. This may allow for the user to control operations of the second endovascular robotic instrument from an external location and allow the user to receive realistic haptic feedback from the first and/or second endovascular robotic instruments.

In some examples, the first endovascular robotic instrument comprises the sensor as described in relation to one or more of the above-specified aspects and examples, and wherein the first endovascular robotic instrument is configured to generate the first haptic feedback data based on the amount of the emitted light detected by the light sensor.

Any advantages and features described in relation to the any of the above aspects and examples may be realized in any of the other aspects and examples described above.

It is clear to a person skilled in the art that certain features of the sensor and/or the system set forth herein may be implemented under use of hardware circuits, software means, or a combination thereof. The software means can be related to programmed microprocessors or a general computer, an ASIC (Application Specific Integrated Circuit) and/or DSPs (Digital Signal Processors). For example, the processing unit may be implemented at least partially as a computer, a logical circuit, an FPGA (Field Programmable Gate Array), a processor (for example, a microprocessor, microcontroller (μC) or an array processor)/a core/a CPU (Central Processing Unit), an FPU (Floating Point Unit), NPU (Numeric Processing Unit), an ALU (Arithmetic Logical Unit), a Coprocessor (further microprocessor for supporting a main processor (CPU)), a GPGPU (General Purpose Computation on Graphics Processing Unit), a multi-core processor (for parallel computing, such as simultaneously performing arithmetic operations on multiple main processor(s) and/or graphical processor(s)) or a DSP.

Even if some of the aspects described above have been described in reference to the arrangement, these aspects may also apply to a method and vice versa.

### BRIEF DESCRIPTION OF THE DRAWINGS

These and other aspects of the invention will now be further described, by way of example only, with reference to the accompanying figures, wherein like reference numerals refer to like parts, and in which:

FIG. 1 shows a cut-away view of a schematic illustration of the sensor according to example implementations as described herein;

FIG. 2 shows a perspective view of a schematic illustration of parts of the sensor according to example implementations as described herein;

FIGS. 3a and 3b show schematic block diagrams of the resilient force member according to example implementations as described herein;

FIG. 4 shows a schematic block diagram of the optical unit according to example implementations as described herein;

FIG. 5 shows a schematic block diagram of the light sensor and the processing unit according to some example implementations as described herein;

FIG. 6 shows a cut-away view of a schematic illustration of a sensor according to some example implementations as described herein;

FIG. 7 shows a perspective view of a schematic illustration of parts of the sensor according to some example implementations as described herein;

FIG. 8 shows a perspective view of a schematic illustration of a gripper mechanism according to some example implementations as described herein;

FIG. 9 shows a schematic block diagram of a procedure of detecting the position of the moveable member according to some example implementations as described herein; and

FIG. 10 shows an endovascular robotic system according to some example implementations as described herein.

### DETAILED DESCRIPTION OF THE PREFERRED EMBODIMENTS

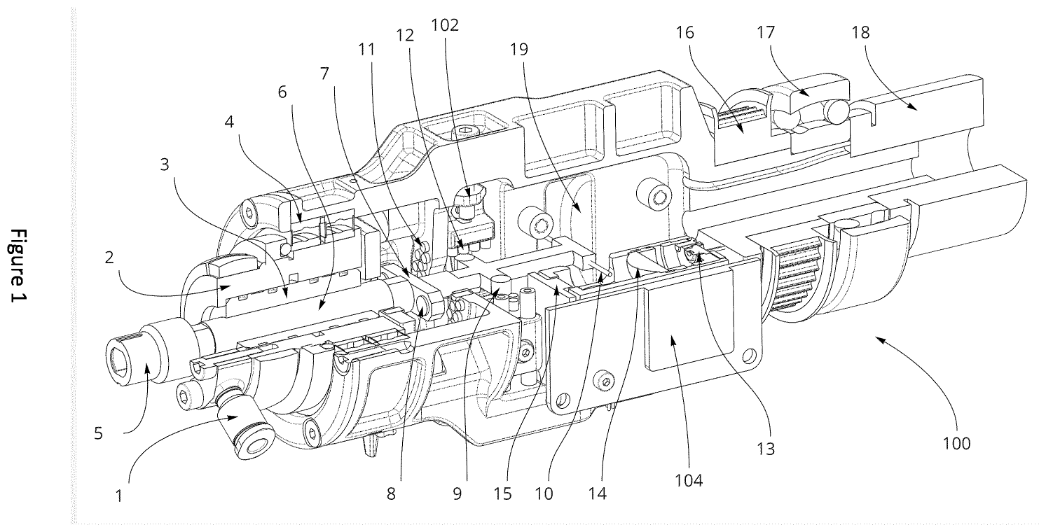

FIG. 1 shows a cut-away view of a schematic illustration of the sensor 100 according to example implementations described herein.

The moveable member 6 extends throughout the sensor. The moveable member 6 may be made of metal, plastic, carbon fiber or any other suitable material. A wire gripper coupling 5 is coupled to the moveable member 6 at one end of the moveable member 6. The wire gripper coupling 5 may allow for an elongated endovascular instrument to be coupled to the moveable member 6. The elongated endovascular instrument may be coupled to the moveable member 6 by any suitable means. The wire gripper coupling 5 may additionally or alternatively couple any suitable instrument to the moveable member 6. In some examples, there is no wire gripper coupling 5 and the moveable member 6 is the instrument itself. The wire gripper coupling 5 may be located at any suitable position on the moveable member 6. Additionally or alternatively, the elongated endovascular instrument may be an endovascular medical instrument such as a guide wire, stent, balloon, catheter, or any other suitable endovasular instrument.

As the moveable member 6 enters the sensor 100, the moveable member 6 moves through an air bearing 3. The air bearing 3 is supplied with air via a compressed air input 1. The air bearing 3 is comprised within an air bearing housing 2. The construction of an air bearing 3 is known to the skilled person. In some examples, the air bearing 3 is replaced by a ball bearing, a roller bearing, a magnetic bearing or any other suitable type of bearing. In some examples, there is more than one type of bearing at the entrance to the sensor 100. In some examples, there is no bearing at the entrance of the sensor 100. The air bearing may be placed at any suitable position of the sensor 100 and/or the moveable member 6.

The moveable member 6 then comprises a plurality of resilient force members which comprise a resilient force unit 102 which is described in further detail below.

At the end of the moveable member 6 opposite to the end which comprises the wire gripper coupling 5, the moveable member 6 comprises a needle 10. The needle 10 comprises two main sections. One section extends radially from the moveable member 6 towards the optical unit 104 which will be described in further detail below. The second section extends radially from the moveable member 6 towards an oscillation dampening pool 19. The oscillation dampening pool 19 may allow for a reduction in unwanted movements of the moveable member 6. The oscillation dampening pool 19 may be of any design and may comprise any fluid which allows for the reduction of oscillations. In some examples, the needle 10 is comprised of two distinct portions coupled via the moveable member 6. In some examples, the needle 10 comprises or is one element which travels through the moveable member 6 and is fixed in place in the moveable member 6 via any suitable means. The needle 10 may be of any suitable design which allows for the operation of the sensor. In some examples, the needle 10 does not extend radially from the moveable member 6 but in any suitable direction.

The sensor further comprises a pulley 16 coupled to a stepper motor 23 which will be described in further detail below.

The sensor comprises a plurality of roller bearings 4, 17. The roller bearings 4, 17 may allow for the housing 20 which encloses the sensor 100 to rotate. The roller bearings 4, 17 surrounding the housing 20 allow the housing 20 to rotate and measure the torque of the rotation of the housing 20 in a dynamic manner during continuous rotation of the housing 20. The rotation of the housing 20 may also allow for a user of the sensor 100 to have finer control over the moveable member 6. The roller bearings 4, 17 may be any suitable bearing such as an air bearing, a ball bearing or a magnetic bearing. The roller bearings 4, 17 maybe coupled to the housing 20 at any suitable location and/or via any suitable method. In some examples, there is only one roller bearing 4, 17. In some examples, there are no roller bearings 4, 17.

The sensor 100 further comprises, in this example, a slip ring 18. In some examples, the slip ring 18 allows for data from the optical unit to be transferred to an external device as will be described in more detail below. In some examples, there is no slip ring 18.

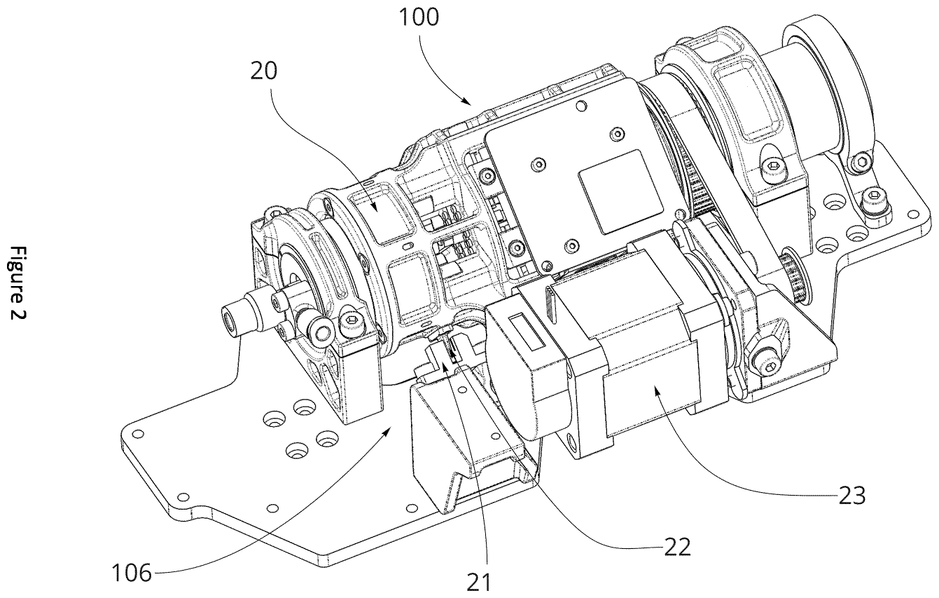

FIG. 2 shows a perspective view of a schematic illustration of parts of the sensor according to example implementations as described herein.

The sensor 100 comprises, in this example, a zero positioning unit 106 and a stepper motor 23.

The zero positioning unit 106 comprises a zero position sensor 21 and a zero position flag 22. When the sensor is in the zero position i.e. in an unbiased position, the zero position sensor 21 indicates that the sensor 100 is in a zero position. The zero position sensor 21 may comprise a light emitting diode and a photodiode. The zero position flag 22 may be coupled to the housing 20 and/or the moveable member 6. The zero position flag 22 may be a protrusion from the moveable member 6 and/or the sensor 100 and/or the sensor housing 20 which is configured to pass through the zero position sensor 21 as the moveable member 6 and/or the sensor 100 and/or the sensor housing 20 is rotated. As the flag 22 is rotated during rotation of the housing 20 and/or the moveable member 6, it will, in certain positions, travel between the light emitting diode and the photodiode. When the flag 22 is situated between the light emitting diode and the photodiode, the photodiode may send a signal to an indicator to indicate the sensor is in the zero position. The indicator may emit light and/or noise and/or any other suitable emission method to indicate to the user that the sensor is in the zero position. The light emitting diode may alternatively be any suitable light emitting device. The photodiode may alternatively be any suitable electronic device which allows for the detection of light emitted from the light emitting device. The flag 22 may be of any suitable design which allows for the indication of the zero position. In some examples, there is no zero positioning unit 106.

The stepper motor 23 may be coupled to the pulley 16 shown in FIG. 1. The stepper motor 23 rotates the sensor housing 20 through the pulley 16 to provide feedback to the user of the sensor 100. For example, if the feedback is set to 0 Nm by a processor of the stepper motor 23, and a torque is applied to the moveable member 6, the stepper motor 23 rotates the sensor housing 20 to counter act the torque exerted by the user and returns the sensor displacement to 0 Nm. The feedback torque may be set to any suitable torque via the processor. In some examples, the pulley 16 is a timing belt.

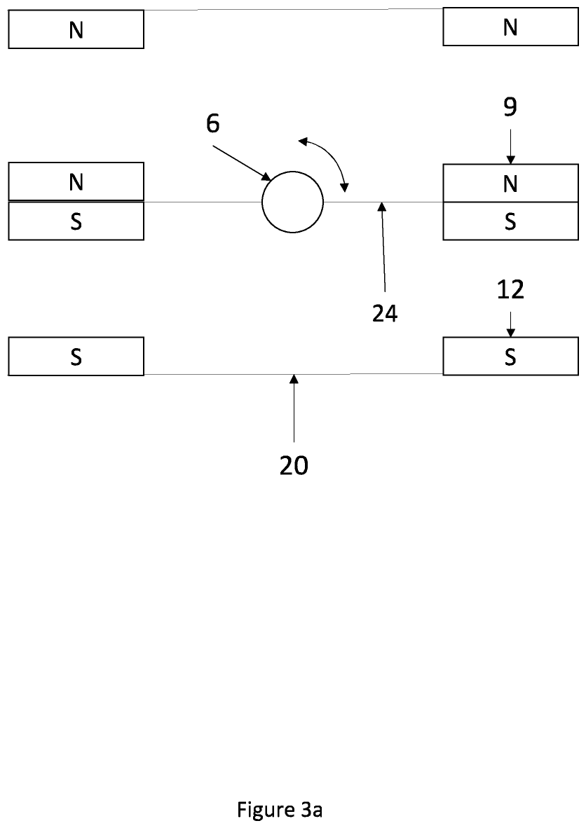

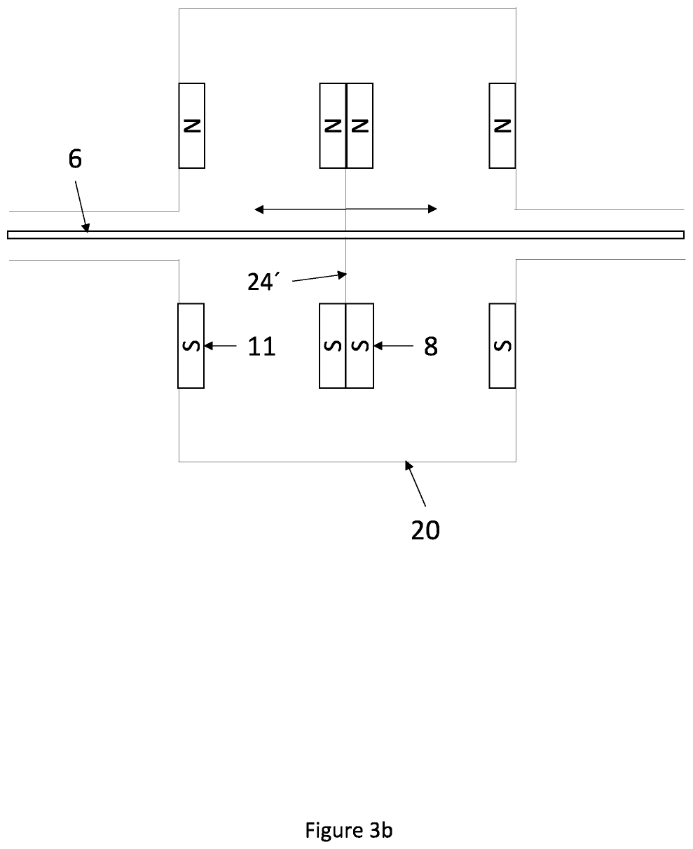

FIGS. 3a and 3b show schematic block diagrams of the resilient force member 24 according to example implementations as described herein, wherein the resilient force member 24 is incorporated in the sensor shown in FIGS. 1 and 2.

FIG. 3a shows a schematic block diagram of the rotational resilient force member 24. In this example, the moveable member 6 comprises a resilient force member 24 which is parallel to the axis of rotation of the moveable member 6. On this resilient force member 24, there are four magnets 9. Each of the magnets 9 on the resilient force member 24 has a singular polarity. In some examples, there are a different number of magnets. In some examples, one or more of the magnets 9 on the resilient force member 24 have a plurality of polarities. A further four magnets 12 are mounted on the housing 20 of the sensor 100. In some examples, these magnets 12 are merely mounted to a section of the sensor 100 which is not the moveable member 6. The magnets 12 mounted to the housing 20 are, in this example, stationary and cannot move during operation of the sensor 100. In this example, each magnet 12 mounted to the housing 20 has a corresponding magnet 9 mounted to the resilient force member 24 in order to create a magnet pair. Each magnet 9, 12 in the magnet pairs has the same polarity so that they repel each other as the moveable member 6 is rotated in the direction of the arrow. This leads to a nonlinear resilient force being felt by the user of the sensor 100. This also allows for the moveable member 6 to be in an unbiased position, i.e. a zero position, when the sensor 100 is not in use. The strength of the magnets may be altered depending on the use of the sensor 100. The distance between the magnets 12 mounted to the housing 20 and the magnets 9 on the resilient force member 24 may be altered via any suitable method such as a screw or a movable platform. In some examples, the strengths of the magnets 9, 12 and/or the distance between the magnets 9, 12 is not the same for each magnet pair. In some examples, the magnets 9, 12 in each magnet pair has opposite polarities.

FIG. 3b shows a schematic block diagram of the linear resilient force member 24′. The linear resilient force member 24′ is constructed in substantially the same way as the rotational resilient force member 24. In this example, the moveable member 6 comprises an resilient force member 24′ which is perpendicular to the longitudinal axis of the moveable member 6 and extends radially from the moveable member 6.

On this resilient force member 24′, there are four magnets 8. Each of the magnets 8 on the resilient force member 24′ has a singular polarity. In some examples, there are a different number of magnets. In some examples, one or more of the magnets 8 on the resilient force member 24′ has a plurality of polarities. A further four magnets 11 are mounted on the housing 20 of the sensor 100. In some examples, these magnets 11 are merely mounted to a section of the sensor 100 which is not the moveable member 6. The magnets 11 mounted to the housing 20 are, in this example, stationary and cannot move during operation of the sensor 100. In this example, each magnet 11 mounted to the housing 20 has a corresponding magnet 8 mounted to the resilient force member 24′ in order to create a magnet pair. Each magnet 8, 11 in the magnet pair has the same polarity so that they repel each other as the moveable member 6 is moved in the direction of the arrow. This leads to a nonlinear resilient force being felt by the user of the sensor. This also allows for the moveable member 6 to be in an unbiased position, i.e. a zero position, when the sensor 100 is not in use. The strength of the magnets may be altered depending on the use of the sensor 100. The distance between the magnets 11 mounted to the housing 20 and the magnets 8 on the resilient force member 24′ may be altered via any suitable method such as a screw or a movable platform. In some examples, the strengths of the magnets 8, 11 and/or the distance between the magnets 8, 11 is not the same for each magnet pair. In some examples, the magnets 8, 11 in each magnet pair have opposite polarities.

The resilient force members 24, 24′ may be of any design which allows for the operation of the sensor 100. The resilient force members 24, 24′ may comprise plastics, metals, carbon fiber or any other suitable material. The resilient force members 24, 24′ described above together comprise the resilient force unit 102 shown in FIG. 1.

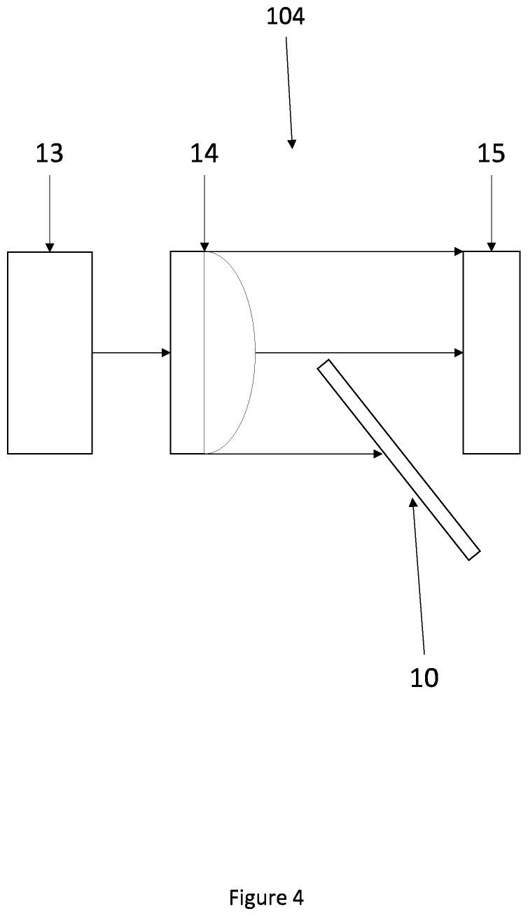

FIG. 4 shows a schematic block diagram of the optical unit according to example implementations as described herein.

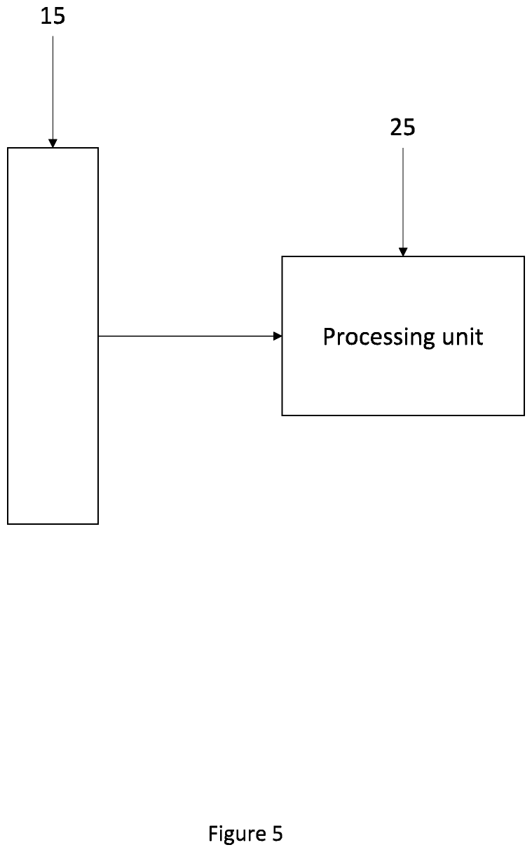

The optical unit 104 comprises, in this example, three main parts, the light source 13, the collimating lens 14, and the light sensor 15. The light source 13 is, in this example, a light emitting diode but may additionally or alternatively be any suitable light emitting device. The light source 13 emits light along an optical path indicated by the arrows in FIG. 4. The optical path then reaches the collimating lens 14. The collimating lens 14 is configured to collimate the light of the optical path. After exiting the collimating lens 14, the optical path reaches the light sensor 15. The light sensor 15 is preferably a linear camera with a 1500×1 pixel array, but may be any suitable light sensor 15. The light sensor 15 senses the light received by said sensor 15 and transmits this data to a processing unit (shown in FIG. 5). The processing unit may comprise a processor, a memory, a transceiver or any other suitable component.

Situated between the collimating lens 14 and the light sensor is the needle 10 (or generally a light-blocking element). The needle 10 moves through the optical path depending on the movement of the moveable member 6 to which the needle 10 is coupled. Due to the needle 10 being in the optical path, a portion of the optical path is blocked. The light sensor 15 is able to sense the amount of light blocked in the optical path by the needle 10 and/or the areas in which the light is blocked in the optical path by the needle 10. The light sensor 15 senses the amount of light not blocked by the needle 10, and the optical unit 104 can calculate the amount of the blocked light by knowing the total amount of light emitted by the light source 13 and the amount of light sensed by the light sensor 15. In some examples, the motion of the moveable member 6 can be based (directly) on the amount of light sensed by the light sensor 15, instead of calculating the amount of light blocked by the needle 10.

FIG. 5 shows a schematic block diagram of the light sensor and the processing unit according to some example implementations as described herein

The light sensor 15 transmits the sensed light data to the processing unit 25. The processor of the processing unit 25 can then process this data to determine which position the needle 10 is in. The processor can also determine changes in received data as the needle 10 is moved from a first position to a second position. Additionally or alternatively, the processor may also be able to calculate the resilient force exerted on the moveable member 6 by processing the received data. Additionally or alternatively, only the amount of light sensed by the light sensor 15 (and/or the amount of light blocked by the needle 10 as calculated based on the amount of light emitted by the light source 13 and the amount of light sensed by the light sensor 15) is used further in order to provide feedback data on a movement of the moveable member 6.

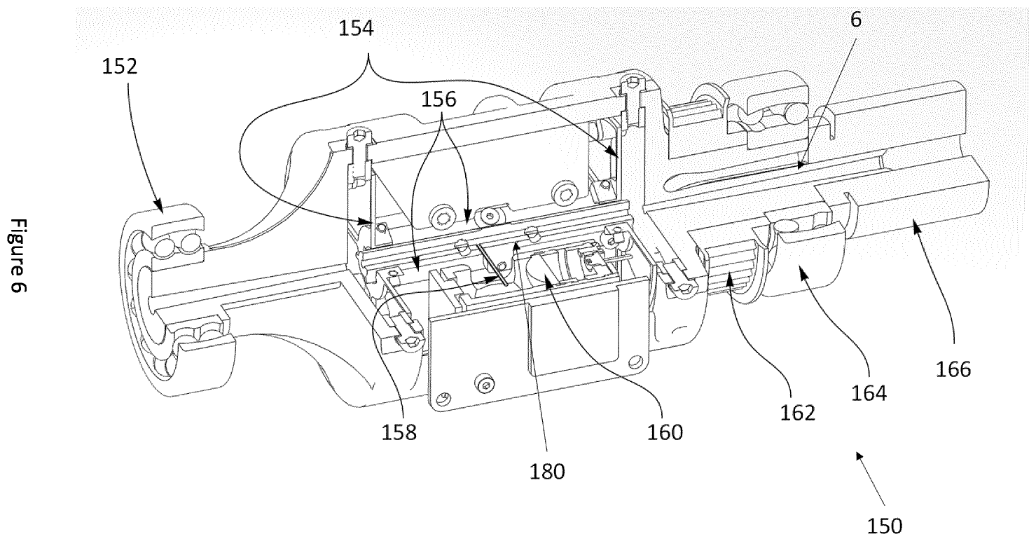

FIG. 6 shows a cut-away view of a schematic illustration of a sensor 150 according to some example implementations as described herein.

The sensor 150 shown in FIG. 6 is a variation of the sensor 100 described above. The sensor 150 of FIG. 6 comprises a front bearing 152, an axial displacement elastic component 154, a rotational displacement elastic component 156, a displacement needle 158, an optical sensor 160, a pulley 162, a rear bearing 164, and a slip ring 166. The resilient force member is, in this example, made up by both of the axial displacement elastic component 154 and the rotational displacement elastic component 156.

The axial displacement elastic component 154 and the rotational displacement elastic component 156 are both located within an elastic component unit 180 which will be described in more detail below.

The resilient force member, the displacement needle 158, the optical sensor 160, the pulley 162 and the slip ring 166 may be (substantially) similar or identical to the respective components described above in relation to FIGS. 1 to 5.

In this example, the front bearing 152 is not an air bearing but is a roller bearing. In some examples, the roller front bearing may be a ball bearing, a magnetic bearing or any other suitable type of bearing which allows for substantially frictionless movement of the moveable member 6. The rear bearing 164 may also be a roller bearing but may alternatively be a ball bearing, a magnetic bearing or any other suitable type of bearing which allows for substantially frictionless movement of the moveable member 6.

The axial displacement elastic component 154 comprises of two parts in this example. The two parts of the axial displacement elastic component 154 are located at two different sections of the sensor and are positioned along the same axis. That is to say, the two parts are both located substantially along the longitudinal axis of the sensor 150.

In this example, the two parts of the axial displacement elastic component 154 comprise four plastic extensions. The four extensions are designed so that they extend from the center of the part in a cross shape i.e. at right angles. The extensions are preferably made of plastic, in particular PETG, but may be made of any other suitable material such as, for example, metal or carbon fiber. In some examples, only a section of one or more of the extensions comprise plastic. There may be any number of extensions in the parts of the axial displacement elastic component 154. The extensions may be located in any suitable position and any suitable orientation within the two parts of the axial displacement elastic component 154. In some examples, the extension design within each part is not identical. In some examples, there is only one part in the axial displacement elastic component 154 and in some examples, there are more than two parts in the axial displacement elastic component 154.

The extensions have a predetermined flexure which allows for them to elastically deform while providing non-linear haptic feedback to the user of the sensor 150. That is to say, when the user “pushes” the moveable member 6, the two parts of the axial displacement elastic component 154 elastically deform to provide the user with a mechanical resistance force. In some examples, the axial displacement elastic component 154 further comprises a limiting member which is configured to prevent the axial displacement elastic component 154 from overdeforming. This may result in a safer sensor 150 with a longer lifespan.

The rotational displacement elastic component 156 performs in a similar way to that of the axial displacement elastic component 154 but in the rotational axis instead of the longitudinal axis.

In this example, the two parts of the rotational displacement elastic component 156 comprise four plastic extensions extending between the two parts of the axial displacement elastic component 154. The four extensions are designed so that they extend from along the longitudinal axis of the sensor in a cross shape i.e. at right angles. The extensions are preferably made of plastic, in particular PETG, but may be made of any other suitable material such as, for example, metal or carbon fiber. In some examples, only a section of one or more of the extensions comprise plastic. There may be any number of extensions in the parts of the rotational displacement elastic component 156. The extensions may be located in any suitable position and any suitable orientation with respect to the two parts of the axial displacement elastic component 154. In some examples, there are fewer than four extensions in the rotational displacement elastic component 156 and in some examples, there are more than four extensions in the axial displacement elastic component 154.

The extensions have a predetermined flexure which allows for them to elastically deform while providing non-linear haptic feedback to the user of the sensor 150. That is to say, when the user rotates the moveable member 6, one or more extensions of the rotational displacement elastic component 156 elastically deform to provide the user with a mechanical resistance force. In some examples, the rotational displacement elastic component 156 further comprises a limiting member which is configured to prevent the rotational displacement elastic component 156 from overdeforming. This may result in a safer sensor 150 with a longer lifespan.

In some examples, one or more extensions of the rotational displacement elastic component 156 are directly coupled to one or more parts of the axial displacement elastic component 154. In some examples, one or more of the extensions of the rotational displacement elastic component 156 are indirectly coupled to one or more parts of the axial displacement elastic component 154. In some examples, one or more extensions of the rotational displacement elastic component 156 are coupled to the housing of the sensor 150.

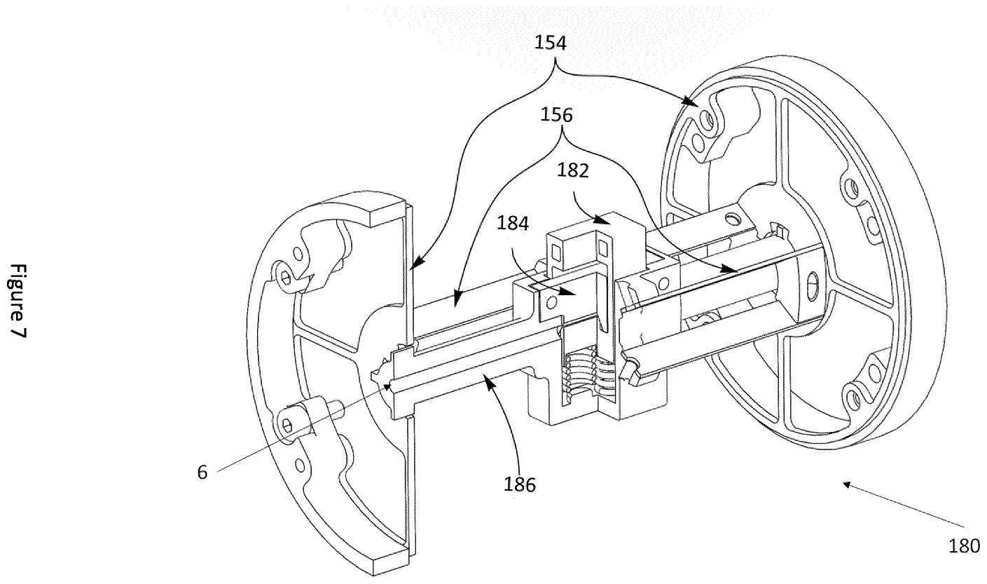

FIG. 7 shows a perspective view of a schematic illustration of parts of the sensor according to some example implementations as described herein.

The elastic component unit 180 comprises the axial displacement elastic component 154 and the rotational displacement elastic component 156 as described above. In some examples, the elastic component unit 180 further comprises the displacement needle 158.

The elastic component unit 180 further comprises an actuated gripper component 182, a static gripper component 184 and a gripper housing 186.

The actuated gripper component 182 may comprise any suitable material such as, for example, plastic, metal or carbon fiber. The actuated gripper component 182 is pushed down via an actuator. The actuator may be a machine or a user pushing down the actuated gripper component 182. By pushing down on the actuated gripper component 182, a one or more springs are compressed. In some examples, there is only a single spring and/or any other suitable elastic component such as, for example, rubber.

The static gripper component 184 is substantially the same as the actuated gripper component 182 but does, in this example, not have the ability to be actuated.

The moveable member 6 is placed inside the gripper housing 186 and through both the actuated and static gripper components 182, 184. When the actuated gripper component 182 is relieved from the actuation motion exerted by a machine or a user, the one or more springs uncompress, pushing the actuated gripper component 182 up, thereby securing and gripping the moveable member 6 in place.

The actuated gripper component 182 may be actuated in any suitable direction depending on the design of the actuated gripper component 182 i.e. from the side or from below.

In some examples, the elastic component unit 180 is replaceable. That is, the elastic component unit 180 is removable from an opening in the sensor 150 and it can be replaced with another elastic component unit 180. This may allow for easy alteration of the elasticity of the axial and rotational displacement elastic components 154, 156 depending on the parameters of the operation the sensor 150 is undertaking.

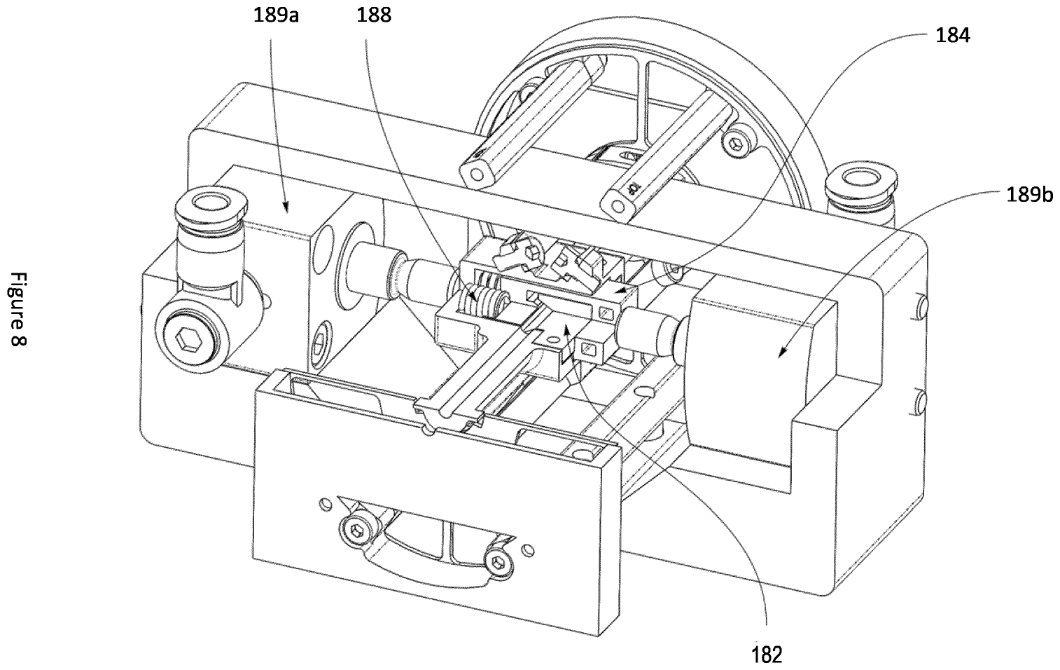

FIG. 8 shows a perspective view of a schematic illustration of a gripper mechanism according to some example implementations as described herein.

The gripper, as described above, comprises an actuated gripper component 182 and a static gripper component 184. The actuated gripper component 182 is biased in a “closed” position, i.e. fixing the moveable member 6 in place, by a pair of springs 188. The springs 188 may be of any suitable strength and comprise any suitable material. In some examples, there is only one spring 188 or more than three springs 188. In some examples, there are no springs 188. In some examples, the springs 188 are replaced by any suitable elastic component. When the actuated gripper component 182 needs to be opened, the first cylindrical member 189a engages with the actuated gripper component 182 to provide support and the second cylindrical member 189b contacts the actuated gripper component 182 in order to force the actuated gripper component 182 into an “open” position i.e. allowing the moveable member 6 to move through the actuated gripper component 182. The cylindrical members 189a, 189b may be of any suitable design such as, for example, prismatic or cuboidal. In this embodiment, the cylindrical members 189a, 189b are pneumatic cylindrical members 189a, 189b. Additionally or alternatively, the cylindrical members 189a, 189b may be moveable via electric motors, a generator, electromagnetic means or any other suitable means.

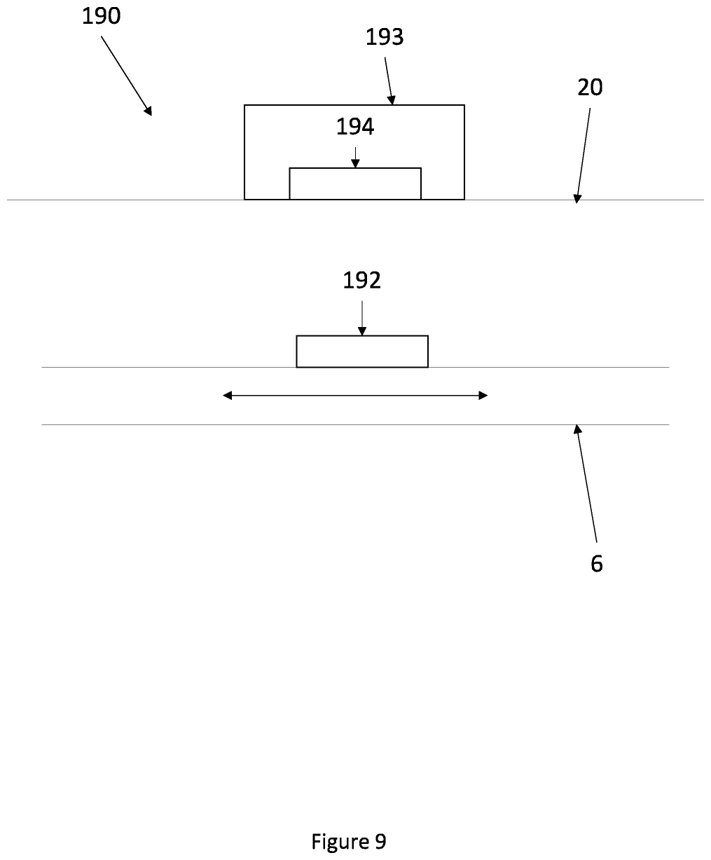

FIG. 9 shows a schematic block diagram of a procedure of detecting the position of the moveable member according to some example implementations as described herein. FIG. 9 shows an alternate method/procedure 190 for detecting the position and/or orientation of the moveable member 6. In this example, a first magnet 192 is directly coupled to the moveable member. A magnetic field measurement unit 193 comprises a second magnet 194 which itself is coupled to the outside of the housing 20 of the sensor 100, 150. This method of detecting the position and/or orientation of the moveable member 6 can be used additionally or alternately to the method involving the displacement needle 10, 158 described above.

The second magnet 194 may be replaced with a Hall effect sensor, in particular one that conforms to the AS5013 international standard. Within the magnetic field measurement unit 193, there is a processing unit which comprises a microcontroller and a transmitter. The coupling between the processing unit and the second magnet 194 is similar to the coupling between the light sensor 15 and the processing unit 25 described above in relation to FIG. 5.

In some examples, the first magnet 192 is not directly coupled to the moveable member 6 but is coupled to an extension of the moveable member 6. In some examples, the magnetic field measurement unit 193 and the second magnet 194 are coupled to the housing 20 but located in the inside of the housing 20. In some examples, the magnetic field measurement unit 193 and the second magnet 194 are coupled to a portion of the sensor which is not the housing 20.

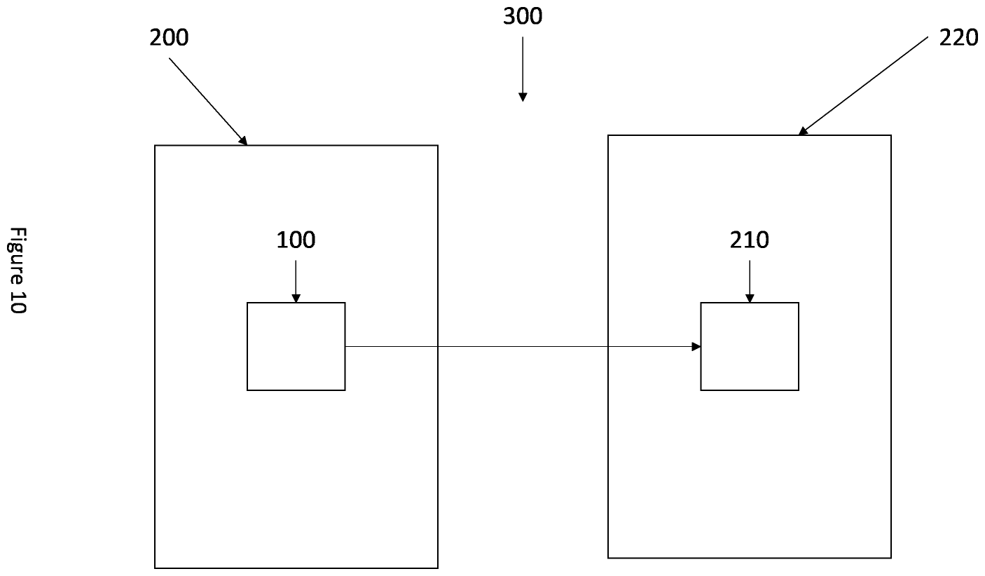

FIG. 10 shows an endovascular robotic system according to some example implementations as described herein.

The processor of the processing unit 25 described above in relation to FIG. 5 then transmits the positional data of the needle 10 to the transceiver of the processing unit 25 which in turn, transmits the data to an external device 210. The external device 210 may be a computer, a server, a device with a sensor similar (or identical) to the sensor 100 described above or any other suitable device. The transceiver may transmit the data via a wired and/or a wireless connection. If the sensor housing 20 is rotatable and the connection is a wired connection, the data by be transmitted via the slip ring 18 which allows for data to be safely transmitted between rotatable and fixed objects. If the data is sent to a device with a sensor similar (or identical) to the sensor 100 described above, the positional data from the sensor 100 described above may be mimicked in the sensor within the external device 210. That is to say, the movements of the moveable member 6 may be translated to a movement of a moveable member of the other sensor, in particular based on data relating to the amount of light blocked by the needle 10 (calculated based on the amount of light emitted by the light source 13 from which the amount of light sensed by the light sensor 15 is subtracted) and/or the amount of light sensed by the light sensor 15. This may allow for a user to perform an operation from an external location. The data is preferably transmitted via a RS232/RS485 physical connection with proprietary protocol, but any suitable method may be used.

In the example implementation of FIG. 9, the external receiver 210 is a transceiver located in a second endovascular robotic instrument 220 and the sensor 100 is located in a first endovascular robotic instrument 200. This creates an endovascular robotic system 300 comprising the sensor 100, the external device 210 and the two endovascular robotic instruments 200, 220. The movement of the moveable member 6 within the sensor 100 is translated to a movement of a moveable member within a second sensor in the second endovascular robotic instrument 220 by the method described above. This may allow for a user to perform an operation from an external location. In some examples, the movement of the moveable member 6 may be translated to a movement of a plurality of moveable members within a respective plurality of sensors in a respective plurality of endovascular robotic instruments.

In some examples, the sensor 100 described above is located on the patient side of an operation. Additionally or alternatively, the sensor 100 (or an additional said sensor) may be located on the surgeon side of an operation.

In some examples, when the sensor 100 is on the patient side, the sensor 100 senses the wire loads inside the patient. The surgeon is then shown the results on a screen and the surgeon controls the guide wire coupled to the moveable member 6 with a joystick. In some examples, the surgeon has a sensor 100 which is identical or substantially identical to the sensor on the patient side and controls the patient side sensor via this identical or substantially identical sensor.

No doubt many other effective alternatives will occur to the skilled person. It will be understood that the invention is not limited to the described embodiments and encompasses modifications apparent to those skilled in the art and lying within the scope of the claims appended hereto.

1.-40. (canceled)

41. A sensor for an endovascular robotic system, wherein the sensor comprises:
a moveable member moveable between a first position and a second position,
a resilient force member coupled to or integral to the moveable member, wherein the resilient force member is configured to provide a resilient force, when the moveable member is in the second position, to bias the moveable member towards the first position, and
a detection unit configured to detect a change in position of the moveable member from the first position to the second position and/or the second position to the first position.

42. The sensor as claimed in claim 41, wherein the detection unit comprises an optical unit comprising a light source for emitting light and a light sensor for detecting the light emitted by the light source,
wherein a first portion of the moveable member is arranged, in the first and/or second position of the moveable member, in an optical path of the emitted light between the light source and the light sensor for at least partially blocking, by the first portion of the moveable member, the emitted light travelling on the optical path between the light source and the light sensor, and
wherein a first amount of the emitted light which is blockable by the first portion of the moveable member in the optical path between the light source and the light sensor is different between the moveable member being in the first position and the moveable member being in the second position, respectively.

43. The sensor as claimed in claim 42, wherein the sensor is configured to determine that the moveable member is in the first position and/or the second position based on a second amount of the emitted light, sensed by the light sensor, not blockable by the first portion of the moveable member in the optical path between the light source and the light sensor when the moveable member is in the first position and/or second position.

44. The sensor (100) as claimed in claim 43, wherein the sensor (100) is configured to determine a magnitude of the resilient force based on the second amount of the emitted light sensed by the light sensor (15).

45. The sensor as claimed in claim 42, wherein the sensor is configured to determine that the moveable member is in
a first transition between the first position and the second position and/or a second
transition between the second position and the first position based on a change of a said amount of the emitted light not blockable by the first portion of the moveable member in the optical path between the light source and the light sensor.

46. The sensor as claimed in claim 41, wherein the resilient force is a nonlinear resilient force, and wherein the nonlinear resilient force is configured to change nonlinearly as the moveable member moves between the first position and the second position and/or between the second position and the first position; and/or
wherein the resilient force is a continuous resilient force, and wherein the continuous resilient force is configured to change continuously as the moveable member moves between the first position and the second position and/or between the second position and the first position.

47. The sensor as claimed in claim 41, wherein the resilient force member comprises a plurality of magnets, wherein a first magnet of the plurality of magnets is coupled to the moveable member, and wherein a second magnet of the plurality of magnets is coupled to a part of the sensor different from the moveable member, and wherein the resilient force comprises a magnetic force between the first magnet and the second magnet.

48. The sensor as claimed in claim 47, wherein the plurality of magnets are arranged such that, in a pair of magnets, the magnets repel or attract each other, and wherein the pair of magnets biases the moveable member towards the first position; and/or
wherein a distance between at least two of the plurality of magnets is configured to change based on a movement of the moveable member, and wherein the change in distance between the at least two of the plurality of magnets is configured to change the resilient force.

49. The sensor as claimed in claim 47, wherein the moveable member is moveable in two degrees of freedom, wherein a first plurality of the plurality of magnets is configured to provide a change in a first directional resilient force in relation to a first degree of freedom as the moveable member is moved between the first position and the second position and/or between the second position and the first position, and wherein a second plurality of the plurality of magnets is configured to provide a change in a second directional resilient force in relation to a second degree of freedom as the moveable member is moved between the first position and the second position and/or between the second position and the first position.

50. The sensor as claimed in claim 41, wherein a first portion of the resilient force member is coupled to the moveable member, and wherein the first portion of the resilient force member is configured to contact a first elastic component coupled to a part of the sensor different from the moveable member, and wherein the resilient force comprises a mechanical resistance force between the first portion of the resilient force member and the first elastic component,
in particular (i) wherein the first portion of the resilient force member and the first clastic component are arranged such that the first portion of the resilient force member and the first elastic component bias the moveable member towards the first position, and/or (ii) wherein the moveable member is moveable in two degrees of freedom and wherein the first portion of the resilient force member and the first elastic component are configured to provide a change in a first directional resilient force in relation to a first degree of freedom as the moveable member is moved between the first position and the second position and/or between the second position and the first position, and wherein a second portion of the resilient force member and a second elastic component are configured to provide a change in a second directional resilient force in relation to a second degree of freedom as the moveable member is moved between the first position and the second position and/or between the second position and the first position.

51. The sensor as claimed in claim 41, wherein a second portion of the moveable member is arranged within an oscillation dampening pool, wherein the oscillation dampening pool is configured to provide oscillation dampening to the moveable member.

52. The sensor as claimed in claim 41, further comprising an air bearing, wherein a third portion of the moveable member is arranged at least partially in the air bearing, and wherein the air bearing is configured to allow the moveable member to move in a substantially frictionless manner.

53. The sensor (100) as claimed in claim 41, further comprising a zero positioning unit (106), wherein the zero positioning unit (106) comprises a positioning sensor (21), a positioning flag (22) and an indicator configured to indicate that the sensor is in a zero position, wherein the moveable member (6) does not encounter a net force in the zero position.

54. The sensor as claimed in claim 41, wherein the detection unit comprises an optical unit comprising a light source for emitting light and a light sensor for detecting the light emitted by the light source,
wherein a first portion of the moveable member is arranged, in the first and/or second position of the moveable member, in an optical path of the emitted light between the light source and the light sensor for at least partially blocking, by the first portion of the moveable member, the emitted light travelling on the optical path between the light source and the light sensor,
wherein a first amount of the emitted light which is blockable by the first portion of the moveable member in the optical path between the light source and the light sensor is different between the moveable member being in the first position and the moveable member being in the second position, respectively,
wherein the moveable member is coupled to or comprises a first endovascular robotic instrument,
wherein the sensor is configured to transmit data relating to sensed light stemming from the light source to an external receiver, wherein the external receiver is comprised in a second endovascular robotic instrument controllable based on the received data.

55. The sensor as claimed in claim 54, further comprising a slip ring, wherein the slip ring is configured to allow for substantially continuous transmission of data corresponding to the sensed light to the external receiver.

56. The sensor as claimed in claim 54, wherein the moveable member is coupled to or comprises a first endovascular robotic instrument, wherein (i) the second endovascular robotic instrument is identical or substantially identical to the first endovascular robotic instrument, and/or (ii) wherein a function of the second endovascular robotic instrument is identical to a function of the first endovascular robotic instrument.

57. The sensor as claimed in claim 41, wherein the detection unit comprises a magnetic field measurement unit configured to detect a change in a magnetic field,
wherein in the first and/or second position of the moveable member, a characteristic of the magnetic field is detectable by the magnetic field measurement unit, and
wherein the characteristic of the magnetic field detectable by the magnetic field measurement unit is a first value when the moveable member is in the first position and a second value when the moveable member is in the second position, wherein the first value is different from the second value.

58. The sensor as claimed in claim 57, wherein the sensor is configured to determine that the moveable member is in the first position and/or the second position based on the characteristic detectable by the magnetic field measurement unit when the moveable member is in the first position and/or second position, in particular wherein the sensor is configured to determine a magnitude of the resilient force based on the characteristic detectable by the magnetic field measurement unit when the moveable member is in the second position;
and/or
wherein the sensor is configured to determine that the moveable member is in a first transition between the first position and the second position and/or a second transition between the second position and the first position based on a change of the characteristic detectable by the magnetic field measurement unit;
and/or
wherein the magnetic field measurement unit is configured to transmit data about the detected characteristic to a microcontroller coupled to the magnetic field measurement unit, in particular wherein the magnetic field measurement unit comprises a first magnet;
and/or
wherein the magnetic field measurement unit comprises a Hall effect sensor, in particular further comprising a second magnet coupled to the moveable member, and wherein the characteristic detectable by the magnetic field measurement unit is based on a magnetic field interaction between the first magnet and the second magnet.

59. A sensor for an endovascular robotic system, wherein the sensor comprises:
a moveable member moveable between a first position and a second position, and
an optical unit comprising a light source for emitting light and a light sensor for detecting the light emitted by the light source,
wherein a portion of the moveable member is arranged, in the first and/or second position of the moveable member, in an optical path of the emitted light between the light source and the light sensor for at least partially blocking, by the portion of the moveable member, the emitted light travelling on the optical path between the light source and the light sensor, and
wherein a first amount of the emitted light which is blockable by the portion of the moveable member in the optical path between the light source and the light sensor is different between the moveable member being in the first position and the moveable member being in the second position, respectively, and
wherein the light sensor is configured to determine that the moveable member is in the first position and/or the second position based on a second amount of the emitted light, sensed by the light sensor, not blockable by the portion of the moveable member in the optical path when the moveable member is in the first position and/or second position.

60. A sensor for an endovascular robotic system, wherein the sensor comprises:
a moveable member moveable between a first position and a second position, and
a magnetic field measurement unit comprising a first magnet, wherein the magnetic field measurement unit is configured to detect a change in a magnetic field,
wherein in the first and/or second position of the moveable member, a characteristic of the magnetic field is detectable by the magnetic field measurement unit,
wherein the characteristic of the magnetic field detectable by the magnetic field measurement unit is a first value when the moveable member is in the first position and a second value when the moveable member is in the second position, wherein the first value is different from the second value, and
wherein the sensor is configured to determine that the moveable member is in the first position and/or the second position based on the characteristic detectable by the magnetic field measurement unit when the moveable member is in the first position and/or second position.
